(19) United States
(12) Patent Application Publication
(10) Pub. No.: US 2024/0166991 A1
(43) Pub. Date: May 23, 2024

(54) CELL CULTURE MEDIUM COMPOSITION CONTAINING SPIRULINA HYDROLYSATE, AND PREPARATION METHOD THEREFOR

(71) Applicant: Korea Institute of Ocean Science & Technology, Busan (KR)

(72) Inventors: Do-Hyung KANG, Jeju-si (KR); Young-Deuk LEE, Jeju-si (KR); Woon-Yong CHOI, Jeju-si (KR); Youn Sik JEONG, Jeju-si (KR); Areumi PARK, Jeju-si (KR); Yeon-Ji LEE, Jeju-si (KR); Tae-Ho KIM, Jeju-si (KR); Won-Kyu LEE, Jeju-si (KR); Yong-Kyun RYU, Jeju-si (KR)

(21) Appl. No.: 18/551,128

(22) PCT Filed: Apr. 1, 2022
(86) PCT No.: PCT/KR2022/004745

§ 371 Date: Sep. 18, 2023

(30) Foreign Application Priority Data
May 24, 2021 (KR) 10-2021-0066419

(51) Int. Cl. C12N 5/00

(52) U.S. Cl. CPC C12N 5/0018; C12N 2533/90; C12N 2537/00

(57) ABSTRACT

The present disclosure relates to a cell culture medium composition and a preparation method therefor. By replacing an animal serum with a Spirulina hydrolysate, cell growth can be induced and cell proliferation can be promoted to a level greater than or equal to that of a medium containing an animal serum, while significantly reducing the added amount of animal serum.

### TECHNICAL FIELD

The present disclosure relates to a cell culture medium composition comprising a Spirulina hydrolysate, and a method for producing same. In more detail, the present disclosure relates to a cell culture medium composition for replacing animal serum added to a cell culture medium with a Spirulina hydrolysate, and a method for producing same.

### BACKGROUND ART

Cell culture is one of the most important foundational technologies for medical, immunological and biological research. The field of application of cell culture is gradually expanding to, e.g., life phenomenon research through human genome analysis and proteomic analysis, stem cell research for the treatment of incurable diseases and regenerative medicine, toxicity and safety testing of natural and physiologically active substances used as food and pharmaceutical raw materials, diagnosis and prevention methods through analysis of the causes of diseases development, new drug development and mechanism analysis through screening of new drug candidates such as antibiotics, anticancer drugs and vaccines, and production of various recombinant peptides and proteins used in medicine and industry. In addition, the importance of the cell culture is becoming more prominent. Cell research is important because cells derived from a living body are recognized as a living body model and are studied instead of living bodies.

The culture of animal cells began with the culture of fertilized eggs of chicks by Roux in 1885, and cell culture techniques for various animal-derived cells have been developed through the efforts of many researchers. The first human-derived immortalized cell line, the HeLa cell line, was established by Gey in 1952, and the foundation for cell research was laid.

A cell culture medium is absolutely necessary to stably culture living cells derived from living organisms on a flat plate in a laboratory. For cell culture, serum is added to a synthetic medium at a concentration of 10 to 20% and used. The synthetic medium comprises various nutrients, vitamins and minerals. Although its constituents have not been fully identified, serum comprises hormones, adhesion factors, and growth factors essential to cells involved in cell growth, division, and differentiation. Serum essential for animal cell culture is isolated from animals such as fetuses, calves, horses, sheep, pigs, dogs, and goats to be used. Fetal bovine serum (FBS) is most commonly used.

The reason why fetal bovine serum is used the most is that a large body with a large amount of blood is required to smoothly supply serum, and cattle are the most suitable among animals raised worldwide. In culturing animal cells, immunoglobulin in serum derived from other species induces an immune rejection response, which causes various side effects. Since fetal bovine serum is isolated from bovine fetuses prior to birth from mother cows, it has only a minimum amount of native immunoglobulin and induces relatively little immune rejection, as compared to serum derived from other animals, and, thus, is most suitable for cell culture.

The process of producing fetal bovine serum for cell culture involves extracting a fetal bovine fetus from the womb of a pregnant mother cow, collecting blood by inserting a needle into the fetal heart, and then separating and using the serum, which are very unethical and non-environmental. In addition, there is a serious problem that, due to the difficulty and scarcity of the production process, industrially producible fetal bovine serum products are priced very high and have a high cost structure. In addition, due to the international community's recommendation to refrain from animal experiments, the growing reliance on cell research is leading to the destabilization of fetal bovine serum supply and the increased burden of research material costs on industry, academia, and research institutes. Accordingly, the US Food and Drug Administration (FDA), the European Medicines Agency (EMEA) of the European Union, and the international community recommend refraining from using fetal bovine serum and developing serum substitutes.

Recently, a serum-free medium has been in the limelight as an alternative to fetal bovine serum. Serum-free medium is a medium for cell culture that does not use serum, which has unethical, non-environmental and high cost problems, and is a cell culture medium that produces various hormones and growth factors essential for cell culture by recombinant protein synthesis technology and adds them to a synthetic medium, thereby making the use of serum unnecessary.

Demand for serum-free media is rapidly increasing domestically and internationally, and dependence thereon is increasing, especially in the pharmaceutical industry market. The reason for this is that, in the case of recombinant protein drugs produced through cell culture, the use of fetal bovine serum results in the unintended synthesis of recombinant proteins with unknown antigens, which may cause potential side effects, and, thus, the process of obtaining approval as a drug is very difficult and expensive, whereas recombinant protein drugs produced through cells cultured in serum-free media have known components, and, thus, there is little concern about side effects and they can produce high-purity recombinant proteins.

However, since the types of cell lines that can be cultured in serum-free media are limited, the process of adapting cell lines for culture is cumbersome, and the addition of recombinant proteins is required instead of using fetal bovine serum. Thus, serum-free media still have high cost problems. In particular, there are limitations in applying serum-free media to general medical, immunological, and biological research fields, except for the field of production of industrial recombinant peptides and proteins, e.g., cell signaling based on cell culture, mechanism analysis, functional and new drug candidate screening, and cytotoxicity and safety testing of raw materials for cosmetics, food, and pharmaceuticals.

The use of serum substitutes or serum-free media cultures is preferred to overcome uncertainties due to uncharacterized nature of serum composition and lot-to-lot variation of serum (Pei et al., Arch Androl. 49(5):331-42, 2003). In addition, for cells, recombinant proteins or vaccines for therapeutic use grown in cell culture, the addition of animal-derived components is undesirable due to potential viral contamination when administered to human beings, Transmissible Spongiform Encephalopathy (TSE) infectious concerns and/or potential immunogenic effects of animal proteins. Serum substitutes have been developed to minimize the effect of fetal bovine serum on cell cultures and to minimize the amount of animal protein used in cultures of human cells. Serum substitutes such as KNOCKOUT™ (Invitrogen, Calif.) are referred to as chemically defined culture media lacking serum and containing nutrients and other proteins essential for cell growth. KNOCKOUT SR™ contains short half-life protein factors, most of which are contained in commercial formulations. KNOCKOUT SR™ cannot be used as a substitute for fetal bovine serum in the plating of feeder cells due to its lack of adhesion factor, thus resulting in improper cell attachment to the formulation. PC-1™ serum-free medium (Lonza, Maryland) is a low-protein, serum-free medium formulated in a specially modified DMEM/F12 medium base, and contains a complete HEPES buffer system with known amounts of insulin, transferrin, fatty acids and proprietary proteins. Transferrin in PC-1 medium has a half-life of 2-4 weeks in solution. Cellgro COMPLETE™ (Cellgro, Va.) is a serum-free low-protein formulation based on a blend of DMEM/F12, RPMI 1640 and McCoy 5A. Cellgro COMPLETE™ does not contain insulin, transferrin, cholesterol, growth or adhesion factors. Cellgro COMPLETE™ contains a mixture of trace elements and high molecular weight carbohydrates, excess vitamins, non-animal protein sources, and bovine serum albumin (1 g/L). Cellgro FREE™ (Cellgro, Va.) is a serum-free, protein-free growth medium that does not contain any hormones or growth factors.

On the other hand, Spirulina sp. is a type of blue-green algae, recognized by WHO as a complete food, super food, etc., and is being used in medical, health, hunger relief programs, etc. NASA is conducting research and development as space food, and it is a biological material whose safety has been recognized by the FDA and the Ministry of Food and Drug Safety. Eco-friendly genus Spirulina (Spirulina sp.) has been reported to promote cell activity and immune system by containing physiologically active substances such as phycocyanin, beta-carotene, Ca-Sp, GLA and Immolina. It is has antioxidant effect of suppressing active oxygen, which is the cause of aging and various diseases, reaches 20 times that of green and yellow vegetables, and has 10 times the calcium of milk, 20 times the beta-carotene of carrots, 50 times the iron of spinach, and 5 times the protein content of eggs per kg, and minerals, vitamins, EPA and natural pigments. That is, it is a nutritionally complete biomaterial. Spirulina sp. has a very fast cell division and cell growth rate, so production per unit area is much higher than terrestrial animals and plants, production costs are very low, as compared to terrestrial animals and plants, and can be cultured even in desert areas with barren environments, and it is easy to collect raw materials after mass cultivation. A domestic market of 80 billion won has already been formed for it. That is, it is a biological resource with great potential for use in medicine, bio research, and industry.

Accordingly, the present inventors confirmed that safe and eco-friendly Spirulina hydrolysate can be used as a serum substitute for cell culture, and developed a cell culture medium containing Spirulina hydrolysate. They confirmed that the medium exhibited a high level of effect, which is similar to or better than those of the existing media containing animal serum, and have completed the present disclosure.

### DISCLOSURE

### Technical Problem

An objective of the present disclosure is to provide a cell culture medium composition capable of stably culturing cells while reducing the amount of animal serum used and a method for manufacturing the same.

Another objective of the present disclosure is to provide a cell culture medium composition for cell culture with excellent ability to induce cell growth and proliferation while reducing the amount of animal serum used and a method for manufacturing the same.

The objectives of the present disclosure are not limited to the objects described above and other objectives will be clearly understood by those skilled in the art from the following description.

### Technical Solution

According to an aspect of the present disclosure, there is provided a method for producing a cell culture medium composition, the method comprising a first step of obtaining a Spirulina extract from Spirulina; a second step of treating the Spirulina extract with a hydrolytic enzyme to prepare a Spirulina hydrolysate; and a third step of filtering and recovering the Spirulina hydrolysate.

The Spirulina may be at least one selected from the group consisting of Spirulina maxima, Spirulina platensis, Spirulina geitleri, Spirulina siamese, Spirulina major, Spirulina subsalsa, Spirulina princeps, Spirulina laxissima, Spirulina curta, Spirulina spirulinoides.

The first step may be, after homogenizing the Spirulina solution, (a) sonicating, followed by extracting at a high temperature and a high pressure, or (b) freeze-thaw dissolving, followed by extracting by sonication.

The hydrolytic enzyme of the second step may have proteolysis.

The hydrolytic enzyme of the second step may be used alone or in combination of two or more.

Two or more kinds of the hydrolytic enzyme may be used sequentially.

The hydrolytic enzyme of the second step may be selected from the group consisting of Alcalase, bromelain, flavourzyme, pancreatin, papain, pepsin, pronase, protamex, trypsin, or a combination thereof.

The third step may be centrifuging the Spirulina hydrolysate, filtering the supernatant, centrifuging the filtrate, separating the supernatant and lyophilizing the same.

According to another aspect of the present disclosure, the present disclosure provides a cell culture medium composition comprising a Spirulina hydrolysate as an active ingredient.

The hydrolysate may be obtained by treating the Spirulina extract with a proteolytic enzyme.

The proteolytic enzyme may be selected from the group consisting of pepsin, pancreatin, trypsin, pronase, papain, or a combination thereof.

The Spirulina hydrolysate may have a concentration of 5 g/L or less.

The cell culture medium composition may comprise the Spirulina hydrolysate in an amount of 0.1 to 20 vol % (v/v).

The cell culture medium composition may comprise serum and Spirulina hydrolysate in an amount of 0.1 to 20 vol % (v/v).

The cell culture medium composition may comprise serum in an amount of 0.1 to 19.9 vol % (v/v).

The cell culture medium composition may comprise serum and Spirulina hydrolysate in a volume ratio (v/v) of 5:5 to 1:9.

The cells may be animal cells, insect cells or plant cells.

### Advantageous Effects

The cell culture medium composition and method for producing same according to the present disclosure have the effect of inducing and promoting the growth of cells to a level greater than or equal to that of a medium containing an animal serum, while significantly reducing the added amount of animal serum.

A conventional Spirulina extract cell culture medium (SACCS) comprises Spirulina protein in the form of a polymer, whereas the cell culture medium composition of the present disclosure comprises Spirulina hydrolysate (SH) in the form of a peptide obtained by hydrolyzing Spirulina protein, thereby showing a greater effect on cell growth and proliferation.

The cell culture medium composition and method for producing same according to the present disclosure have the effect of reducing infectious diseases derived from animals and reducing environmental and ethical problems occurring in the process of obtaining animal serum.

The effects of the present disclosure are not limited thereto and it should be understood that the effects include all effects that can be inferred from the configuration of the present disclosure described in the following specification or claims.

### DESCRIPTION OF DRAWINGS

FIG. 1 is a process flow chart of a method for preparing a medium composition for cell culture according to the present disclosure.

FIG. 2 shows a process of sequentially treating three hydrolytic enzymes (pepsin, trypsin, and Alcalase) after preparing SH1 and SH2 in the method for producing a cell culture medium composition according to an embodiment of the present disclosure.

FIG. 3A compares SH1 and SH2 with general components of FBS according to an embodiment of the present disclosure. FIG. 3B compares SH1 and SH2 with minerals of FBS. FIG. 3C compares SH1 and SH2 with heavy metals of FBS. FIG. 3D comprises SH1 and SH2 amino acids of FBS. FIG. 3E comprises SH1 and SH2 amino acid ratios of FBS.

FIG. 4 is a SEM image of Spirulina on each step of the method for producing a cell culture medium composition according to an embodiment of the present disclosure.

FIG. 5 shows the survival rate of H460 cells according to the concentrations of SH1 and SH2 according to an embodiment of the present disclosure.

FIG. 6A shows the cell growth rate in a medium supplemented with 3% FBS and 7% Spirulina hydrolysates (SH1-A, SH1-B, SH2-A, SH2-B) according to an embodiment of the present disclosure. FIG. 6B shows the cell survival rate in the medium supplemented with 3% FBS and 7% Spirulina hydrolysate (SH1-A, SH1-B, SH2-A, SH2-B). FIG. 6C shows the cell growth rate in the medium supplemented with 1% FBS and 9% spirulina hydrolysates (SH1-A, SH1-B, SH2-A, SH2-B). FIG. 6D shows the cell survival rate in the medium supplemented with 1% FBS and 9% Spirulina hydrolysates (SH1-A, SH1-B, SH2-A, SH2-B).

FIG. 7 shows the observation of the morphology of H460 cells cultured in a medium supplemented with 3% FBS and 7% Spirulina hydrolysate according to an embodiment of the present disclosure.

FIG. 8 shows the observation of the morphology of H460 cells cultured in a medium supplemented with 1% FBS and 9% Spirulina hydrolysate according to an embodiment of the present disclosure.

FIG. 9 shows a process of treating each of 11 hydrolytic enzymes in the method for producing a cell culture medium composition according to an embodiment of the present disclosure.

FIG. 10 shows the cell growth rates of H460 cells of Spirulina hydrolysates for each type of hydrolytic enzyme according to an embodiment of the present disclosure.

FIG. 11 is a method for producing a cell culture medium composition according to an embodiment of the present disclosure, and shows a process for producing three types of Spirulina hydrolytic enzymes (PA, PB, PC) according to a combination of hydrolytic enzymes.

FIG. 12A shows the cell growth rate of H460 cell line in a medium supplemented with 5% FBS and 5% Spirulina hydrolysate (PA, PB, PC) according to an embodiment of the present disclosure. FIG. 12B shows the cell survival rate of the H460 cell line in a medium supplemented with 5% FBS and 5% Spirulina hydrolysates (PA, PB, PC). FIG. 12C shows the cell growth rate of the H460 cell line in a medium supplemented with 3% FBS and 7% Spirulina hydrolysates (PA, PB, PC). FIG. 12D shows the cell survival rate of the H460 cell line in a medium supplemented with 3% FBS and 7% Spirulina hydrolysates (PA, PB, PC). FIG. 12E shows the cell growth rate of the H460 cell line in a medium supplemented with 1% FBS and 9% Spirulina hydrolysates (PA, PB, PC). FIG. 12F shows the cell survival rate of the H460 cell line in a medium supplemented with 1% FBS and 9% spirulina hydrolysates (PA, PB, PC).

FIG. 13A shows the observation of the morphology of the H460 cell line cultured in a medium supplemented with FBS and Spirulina hydrolysate at a ratio of 5:5 according to an embodiment of the present disclosure. FIG. 13B shows the observation of the morphology of the H460 cell line cultured in a medium supplemented with FBS and Spirulina hydrolysate at a ratio of 3:7.FIG. 13C shows the observation of the morphology of the H460 cell line cultured in a medium supplemented with FBS and Spirulina hydrolysate at a ratio of 1:9.

FIG. 14A shows the cell growth rate of Hela cell line in a medium supplemented with 5% FBS and 5% Spirulina hydrolysates (PA, PB, PC) according to an embodiment of the present disclosure. FIG. 14B shows the cell survival rate of Hela cell lines in a medium supplemented with 5% FBS and 5% Spirulina hydrolysates (PA, PB, PC). FIG. 14C shows the cell growth rate of Hela cell line in the medium supplemented with 3% FBS and 7% Spirulina hydrolysate (PA, PB, PC). FIG. 14D shows the cell survival rate of Hela cell lines in a medium supplemented with 3% FBS and 7% Spirulina hydrolysates (PA, PB, PC).

FIG. 15A shows the observation of the morphology of Hela cell lines cultured in a medium supplemented with FBS and Spirulina hydrolysate at a ratio of 5:5 according to an embodiment of the present disclosure. FIG. 15B shows the observation of the morphology of Hela cell lines cultured in a medium supplemented with FBS and Spirulina hydrolysate at a ratio of 3:7.

### BEST MODE

Hereinafter, preferred embodiments of the present disclosure will be described with reference to accompanying drawings.

The advantages and features of the present disclosure, and methods of achieving them will be clear by referring to the exemplary embodiments that will be described hereafter in detail with reference to the accompanying drawings.

However, the present disclosure is not limited to the exemplary embodiments described hereafter and may be implemented in various ways, and the exemplary embodiments are provided to complete the description of the present disclosure and let those skilled in the art completely know the scope of the present disclosure and the present disclosure is defined by claims.

Further, when it is determined that well-known technologies, etc. may make the scope of the present disclosure unclear, they will not be described in detail in the following description.

Hereinafter, the present disclosure is described in detail.

### Method for Producing a Cell Culture Medium Composition

The present disclosure provides a method for producing a cell culture medium composition, the method comprising a first step of obtaining a Spirulina extract from Spirulina; a second step of treating the Spirulina extract with a hydrolytic enzyme to prepare a Spirulina hydrolysate; and a third step of filtering and recovering the Spirulina hydrolysate.

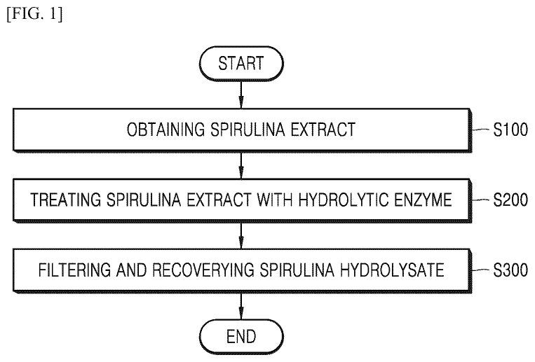

Hereinafter, a method for producing the cell culture medium composition of the present disclosure described with reference to FIG. 1.

First step: Spirulina extract is obtained from Spirulina S100.

In this step, an extract may be obtained by, after homogenizing the Spirulina solution, (a) sonicating and then extracting the same at a high temperature and a high pressure, or (b) freeze-thaw dissolving and then extracting the same by sonication.

The Spirulina is preferably at least one selected from the group consisting of Spirulina maxima, Spirulina platensis, Spirulina geitleri, Spirulina siamese, Spirulina major, Spirulina subsalsa, Spirulina princeps, Spirulina laxissima, Spirulina curta, Spirulina spirulinoides.

The form of the Spirulina is not particularly limited, but is preferably in powder form.

Methods such as ultrasonic waves, lysing enzymes, freeze-thaw dissolution, heating, pressurization, compression, and bead milling may be used in cell membrane disruption, but the present disclosure is not limited thereto.

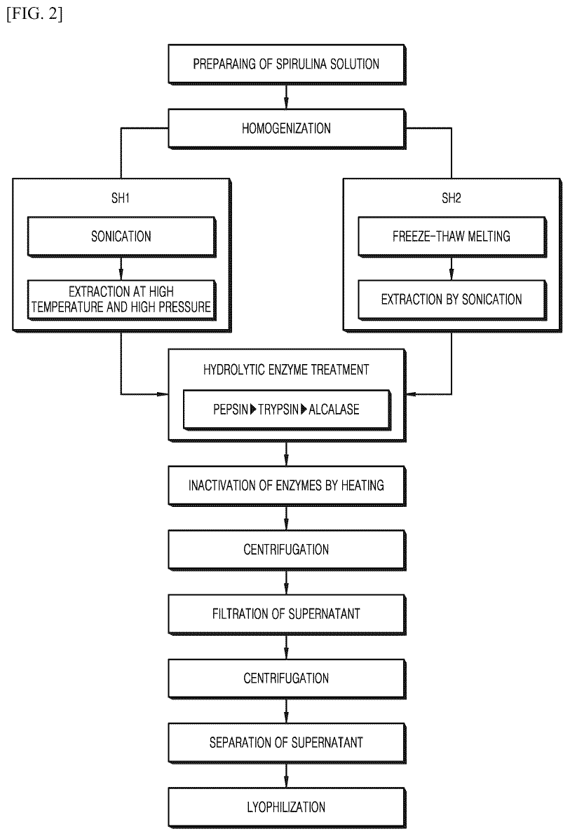

In one embodiment of the present disclosure, Spirulina powder is mixed with a solvent to prepare a Spirulina solution, homogenized, and then (a) the cell membrane is disrupted using ultrasonic waves, and then heat and pressure is applied to obtain a Spirulina extract (FIG. 2, SH1), or (b) freeze-thawing is repeated to dissolve the cell membrane and sonicated to extract Spirulina (FIG. 2, SH2).

Second step: A hydrolysate is prepared by treating the Spirulina extract with a hydrolytic enzyme S200.

In this step, the Spirulina extract is treated with a hydrolytic enzyme such that the hydrolysis yield is improved and damage to the active ingredient is minimized. In the case of hydrolysis through strong acid or high temperature treatment, problems arise in that vitamins are destroyed or sensitive amino acids such as tryptophan are damaged. On the other hand, hydrolysates using enzymes have advantages over acid hydrolysates in that they have a higher yield and less damage to active ingredients.

The hydrolytic enzyme may have proteolytic ability.

The hydrolytic enzyme may be used alone or in combination of two or more.

The hydrolytic enzyme may be one selected from the group consisting of Alcalase, bromelain, flavourzyme, pancreatin, papain, pepsin, pronase, protamex, trypsin, or a combination thereof, but the present disclosure is not limited thereto.

According to one embodiment of the present disclosure, Alcalase, bromelain, flavourzyme, pancreatin, papain, pepsin, pronase, protamex, trypsin, pepsin+trypsin+alcalase, pepsin+pancreatin, pepsin+trypsin, or pepsin+pancreatin+papain were used as hydrolytic enzymes (FIG. 2, 9, 11).

In the case of using two or more types of hydrolytic enzymes in combination as described above, since the reaction temperature and pH of each hydrolytic enzyme are different, it is preferable to use the hydrolytic enzymes sequentially such that each of the hydrolytic enzymes reacts under optimal conditions (reaction temperature, pH, time, etc.). For example, when three types of hydrolytic enzymes are used in combination as shown in FIG. 2, (1) pepsin is added to the Spirulina extract and reacted at 37° C., pH 2 for 2 hours, (2) trypsin is reacted at 37° C., pH 8 for 2 hours, and (3) alcalase is reacted at 50° C., pH 8 for 2 hours to prepare a Spirulina hydrolysate.

After the reaction of each hydrolytic enzyme, it is preferable to inactivate the enzymes by applying heat.

Third step: The Spirulina hydrolysate is filtered and recovered S300.

This step is a step of filtering and recovering the Spirulina hydrolysate, wherein the Spirulina hydrolysate in Step 2 may be centrifuged, and the supernatant may be filtered and then lyophilized. In addition, the filtrate may be centrifuged and then the supernatant may be lyophilized.

The lyophilized Spirulina hydrolyzed powder may be dissolved in distilled water, filtered through a filter to remove contaminants, and then added to a basal medium for cell culture.

The basic medium for cell culture comprises Minimum Essential Medium (MEM), Dulbecco's Modified Eagle Medium (DMEM), DMEM-F12, Roswell Park Memorial Institute (RPMI), Keratinocyte Serum Free Medium (K-SFM), M199, Ham's F12, Ham's 10, NCTC 109, NCTC 135, NeuroCult Basal Medium, etc., and other media are sufficient as long as used in the industry. Further, the medium may comprise at least one selected from the group consisting of various nutrients, minerals, minerals, vitamins, amino acids, recombinant proteins including peptides, attachment factors, growth factors, and hormones, but the present disclosure is not limited thereto.

Preparation of the cell culture medium composition of the present disclosure may be performed by additionally using or applying materials and methods commonly used in the technical field of the present disclosure.

### Cell Culture Medium Composition

The present disclosure provides a cell culture medium composition comprising a Spirulina hydrolysate as an active ingredient.

The cell culture medium composition of the present disclosure is characterized in that it comprises a Spirulina hydrolysate. In contrast to the conventional Spirulina extract cell culture medium (SACCS) comprising a Spirulina protein in the form of a polymer, the cell culture medium composition of the present disclosure comprises a Spirulina hydrolysate (SH) in the form of a peptide obtained by hydrolyzing a Spirulina protein, thereby showing a greater effect on cell growth and proliferation.

Spirulina genus (Spirulina sp.), a kind of blue-green algae, is a raw material notified by the FDA and the Ministry of Food and Drug Safety as a super food and complete food, and its eco-friendliness, nutritional excellence and stability have been proven. Spirulina shows a protein content of 50% or more and contains 6 to 9% of lipids, 15 to 20% of carbohydrates, and various physiologically active substances such as vitamins and minerals.

The Spirulina may comprise at least one selected from Spirulina maxima, Spirulina platensis, Spirulina geitleri, Spirulina siamese, Spirulina major, Spirulina subsalsa, Spirulina princeps, Spirulina laxissima, Spirulina curta, Spirulina spirulinoides.

The hydrolysate is preferably obtained by treating Spirulina with a proteolytic enzyme. In the case of hydrolysis through strong acid or high temperature treatment, problems arise in that vitamins are destroyed or sensitive amino acids such as tryptophan are damaged. On the other hand, the hydrolysate hydrolyzed using an enzyme has an advantage in that the yield is superior to that of the hydrolysate hydrolyzed using an acid and damage to active ingredients is less.

The proteolytic enzyme may comprise one selected from Alcalase, bromelain, flavourzyme, pancreatin, papain, pepsin, pronase, protamex, trypsin, or a combination thereof, but the present disclosure is not limited thereto.

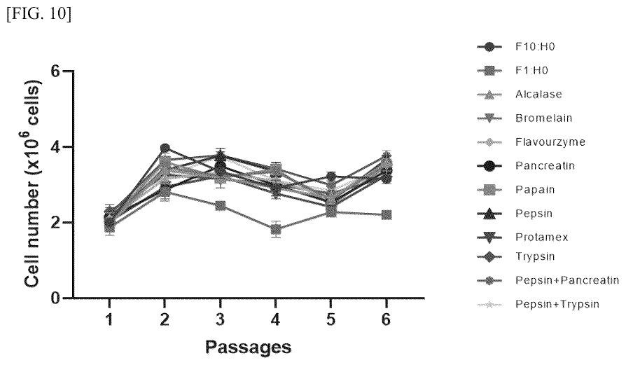

According to one embodiment of the present disclosure, Alcalase, bromelain, flavourzyme, pancreatin, papain, pepsin, pronase, protamex, trypsin, pepsin+trypsin+alcalase, pepsin+pancreatin, pepsin+trypsin, or pepsin+pancreatin+papain were used as hydrolytic enzymes (FIG. 2, 9, 11). Among them, in particular, pepsin+pancreatin, pepsin, pepsin+trypsin, pronase, and papain hydrolysate showed high cell growth rates in that order (FIG. 10).

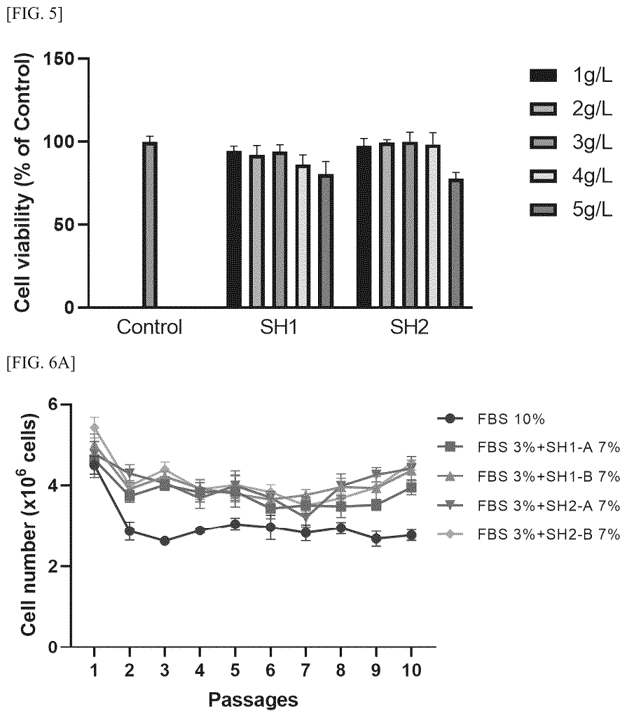

The Spirulina hydrolysate is preferably contained in a concentration of 5 g/L or less. At its concentrations exceeding 5 g/L, there may be a problem that it shows cytotoxicity (FIG. 5).

The cell culture medium composition may further comprise serum, and may comprise serum and Spirulina hydrolysate at 0.1 to 20 vol % (v/v), preferably at 1 to 15 vol % (v/v), and more preferably 5 to 10 vol % (v/v).

The cell culture medium composition may comprise serum in an amount of 0.1 to 19.9 vol % (v/v), preferably 1 to 15 vol % (v/v), and more preferably 5 to 10 vol % (v/v).

The serum may be animal blood, but is not limited thereto, and is preferably serum derived from mammalian (e.g., pig, horse, cow, goat, sheep and dog) blood, and more preferably fetal bovine serum (FBS).

The cell culture composition may be used by adding an appropriate amount of the Spirulina hydrolysate and/or animal serum according to the present disclosure to a basal medium for cell culture. The basic medium for cell culture comprises Minimum Essential Medium (MEM), Dulbecco's Modified Eagle Medium (DMEM), DMEM-F12, Roswell Park Memorial Institute (RPMI), Keratinocyte Serum Free Medium (K-SFM), M199, Ham's F12, Ham's 10, NCTC 109, NCTC 135, NeuroCult Basal Medium, etc., and other media are sufficient as long as used in the industry. Further, the medium may comprise at least one selected from the group consisting of various nutrients, minerals, minerals, vitamins, amino acids, recombinant proteins including peptides, attachment factors, growth factors, and hormones, but the present disclosure is not limited thereto.

The cell culture medium composition preferably comprises serum and spirulina hydrolysate in a volume ratio (v/v) of 5:5 to 1:9. The volume ratio (v/v) of serum to Spirulina hydrolysate may be 5:5, 4:6, 3:7, 2:8, or 1:9, and preferably 5:5, 3:7, or 1:9. As can be seen in FIG. 5, addition of the Spirulina hydrolysate at too high a concentration has a disadvantage that the cell survival rate is lowered and also has a problem that the cell growth rate is significantly reduced in the ratio of FBS 0:SH 10.

The type of cells in which the cell culture medium composition can be used is not particularly limited, and the cell culture medium may be used for culturing animal cells, insect cells, or plant cells, and particularly preferably for culturing human-derived cells. Human-derived cells that may be used in the cell culture medium composition comprise H460, Hela, HCT116, T24, IMR90, HEK293, H1299, and H358 cell lines, but the present disclosure is not limited thereto.

The cell culture medium composition may be subcultured, and may be subcultured for 10 or more generations.

### MODE FOR INVENTION

Hereafter, preferred embodiments are proposed to help understand the present disclosure, but the following embodiments just exemplify the present disclosure and the scope of the present disclosure is not limited to the following embodiments.

### EMBODIMENT

### Embodiment 1

### Preparation of Spirulina Hydrolysate

Two extraction processes were used to prepare a Spirulina hydrolysate (SH) promoting cell proliferation.

As shown in FIG. 2, dried Spirulina powder was dissolved in distilled water at a concentration of 4% and homogenized. Then, it was divided into two parts, and one side was sonicated for 30 minutes to disrupt the cells, and then heat and pressure were applied at 121° C. for 30 minutes to extract SH1. The other side was frozen and thawed at −50° C. and 37° C., which was repeated three times, to disrupt the cells and sonicated for 2 hours to extract SH2.

For protein hydrolysis, extracts SH1 and SH2 were sequentially treated with three hydrolases, pepsin (Sigma Aldrich), trypsin (Sigma Aldrich), and alcalase (Sigma Aldrich) to prepare hydrolysates. The hydrolysis was performed by using an enzyme in 1% (w/w) of the dry weight of Spirulina. Each of Spirulina extracts SH1 and SH2 was added with pepsin and reacted at 37° C., pH 2 for 2 hours, and trypsin was then reacted at 37° C., pH 8 for 2 hours. Finally, alcalase was reacted at 50° C. and pH 8 for 2 hours to prepare a hydrolysate. After each enzyme reaction, the enzyme was inactivated by boiling at 95° C. for 10 minutes, and the pH for optimal enzyme conditions was titrated using HCl and NaOH.

The hydrolysate was centrifuged at 9,000 rpm for 20 minutes, and only the supernatant was taken, filtered using 1 μm filter paper (Whatman, Maidstone, England), and centrifuged at 30,000 rpm for 20 minutes to remove fine residues, and only the supernatant was separated and lyophilized.

The lyophilized Spirulina hydrolyzed powder was dissolved in distilled water before use in the experiment, filtered with a 0.2 μm polyethersulfone (PES) filter (Merck Millipore, Massachusetts, USA) to remove contaminants, and then added to the medium for use.

### Embodiment 2

### Comparison of Components of Hydrolysates According to Extraction Method

The analysis of the general ingredients of SH1 and SH2 hydrolysates was performed according to the Food Code (2015) and AOAC method, and crude fat was analyzed by ether extraction method, and crude protein was analyzed by Kjeldahl method. The analysis of minerals was performed by taking 1 g of the sample in an incineration container and carbonizing and heating same at 550° C. for several hours to incinerate until white to off-white ash was obtained. This ash was sequentially decomposed with hydrochloric acid, diluted to a certain amount, filtered and quantified using an ICP analyzer (Optima8300, Perkin Elmer).

The analysis of lead and cadmium was performed by taking the sample in a crucible, drying and carbonizing same and then incinerating same at 450 to 550° C., wetting the ash with water, adding 2 to 4 mL of hydrochloric acid and drying same on a water bath, and adding 4% nitric acid and heat to dissolve same. The insoluble matter, if present, was filtered through a glass filter, and then diluted to 20 mL and used as a test solution. Lead and cadmium standard solutions, test solutions, and blanks were injected into ICP-OES (Varian, MPX, AUS) and analyzed. The standard solution was prepared by diluting the standard 1000 mg/L standard material with 4% nitric acid to prepare a mixed 100 mg/L stock solvent, and the standard material used in the test was diluted again with 4% nitric acid and used as the standard material.

The analysis of amino acids was performed by the AccQTag amino acid analysis method (Waters). HPLC (Waters 2695, USA) was used as the analysis conditions, AccQTag (3.9×150 mm) was used as a column, and a fluorescence detector (EX: 250 nm, EM: 395 nm) was used for detection.

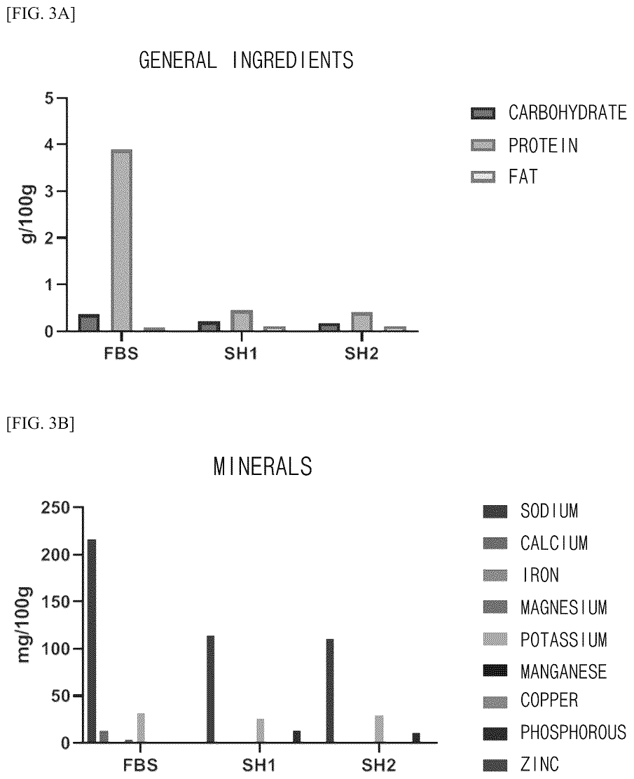

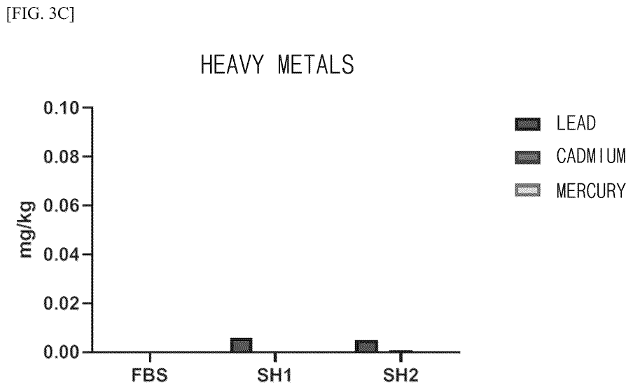

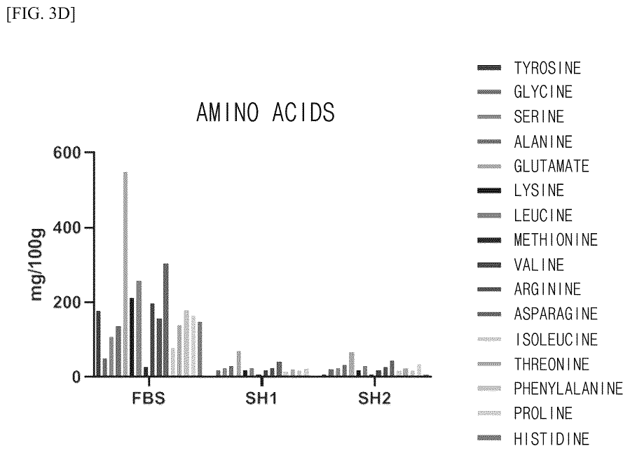

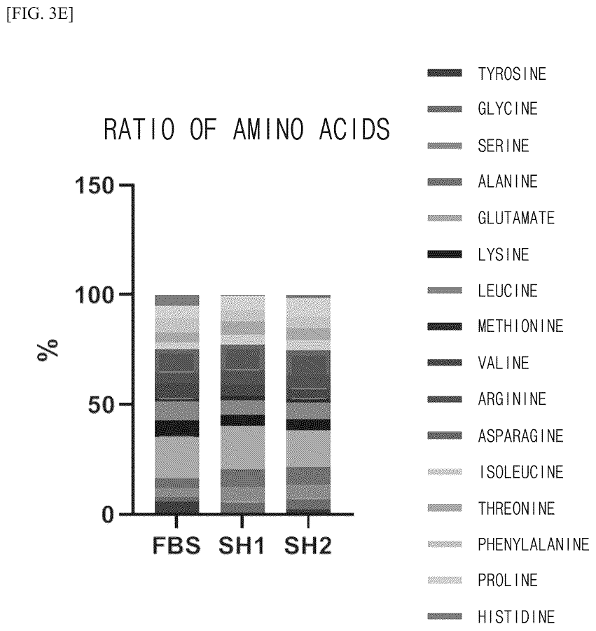

Referring to FIGS. 3A to 3E , as a result of the analyses, there was no significant difference between SH1 and SH2, but the contents of carbohydrates and proteins in SH1 were higher by 0.3 to 0.4 g than those in SH2. In terms of the content of minerals, SH2 had slightly higher contents of potassium and zinc than SH1. Almost no heavy metals were contained therein. The ratio of amino acids was similar, and the content of amino acids in SH1 was higher in glutamate and methionine than those in SH2.

### Embodiment 3

### Analysis of SEM Image for Each Step of Producing Spirulina Hydrolysate

SEM images were taken using a field emission scanning electron microscope (Tescan, Brno, Czechia) at each step of producing a hydrolysate obtained by extracting Spirulina by high temperature and high pressure extraction (SH1) method and hydrolyzing same.

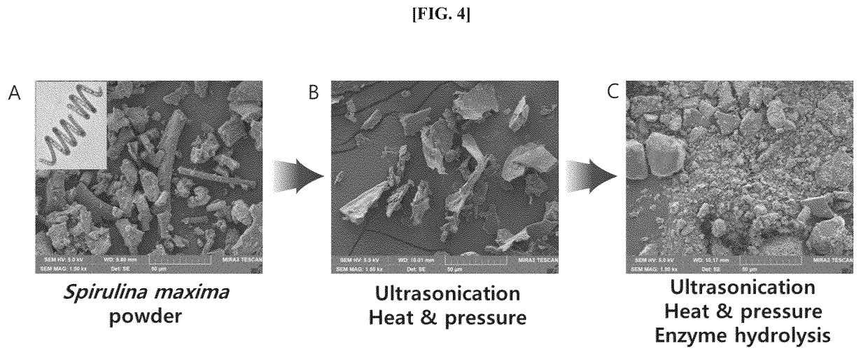

Referring to FIG. 4, it was confirmed that (A) rod-shaped cells remained intermittently in the Spirulina dry powder, (B) Spirulina was destroyed after sonication and high-temperature and high-pressure treatment, and (C) the Spirulina destroyed by enzymatic treatment after sonication and high-temperature and high-pressure treatment was completely decomposed and existed in the form of fine particles.

### Embodiment 4

### Evaluation of Cytotoxicity of Spirulina Hydrolysates

In order to confirm the appropriate concentration of Spirulina hydrolysate for promoting cell growth, cell survival rate was measured according to the concentration of the hydrolysate in a human lung cancer tissue-derived cell line (H460). The SH1 and SH2 hydrolysates prepared in Example 1 were prepared as a stock solution at a concentration of 10 to 50 g/L and used for cell survival rate measurement experiments. The control group was a medium added with 10% FBS.

The cell survival rate was measured using the EZ-CyTox kit (Daelillab, Seoul, Korea), which measures the activity of mitochondrial dehydrogenase in cells. For the culture of the H460 cell line, Roswell Park Memorial Institute (RPMI) 1640 medium (Gibco, Grand Island, USA) and Minimum Essential Medium (MEM) (Sigma Aldrich) added with 10% FBS were used and cultured at 37° C. and 5% CO₂ conditions.

150 μL of the cultured cells were dispensed into a 96-well plate at concentrations of 5.1×103 and 3.7×103 cell/well, respectively, and after 24 hours, 15 μL of each of SH1 and SH2 hydrolysate stock solutions was added to obtain a final concentration of 1 to 5 g/L, incubated for 48 hours in a 37° C., 5% CO₂ incubator, added with 10 μL of EZ-CyTox reagent and cultured for 3 hours, and absorbance at 450 nm was then measured using a plate reader (Biotek, Winooski, USA).

Referring to FIG. 5, no toxicity was observed in the H460 cells at concentrations of 3 g/L or less of SH1 hydrolysate and 4 g/L or less of SH2 hydrolysate.

### Embodiment 5

### Cell Proliferation Effects of Spirulina Hydrolysates According to Extraction Method

In order to confirm the cell proliferation promoting effect of the SH1 and SH2 hydrolysates, H460 cell line was cultured in RPMI 1640 medium at 37° C. and 5% CO₂ conditions. The cultured cells were seeded in a 60 mm cell culture dish at a concentration of 2.0×10⁵ cell/dish.

The SH1 and SH2 hydrolysate stocks (SH1-A: SH1 at a concentration of 10 g/L, SH1-B: SH1 at a concentration of 20 g/L, SH2-A: SH2 at a concentration of 10 g/L, SH2-B: SH2 at a concentration of 20 g/L) were added at 7% and 9%, respectively, to a medium added with FBS at concentrations of 3% and 1% based on a medium added with 10% FBS, and H460 cells were then subcultured for 10 generations. The cell proliferation was observed by measuring the number of cells for each generation, and the cell survival rate was measured using EZ-CyTox reagent after culturing for 10 generations.

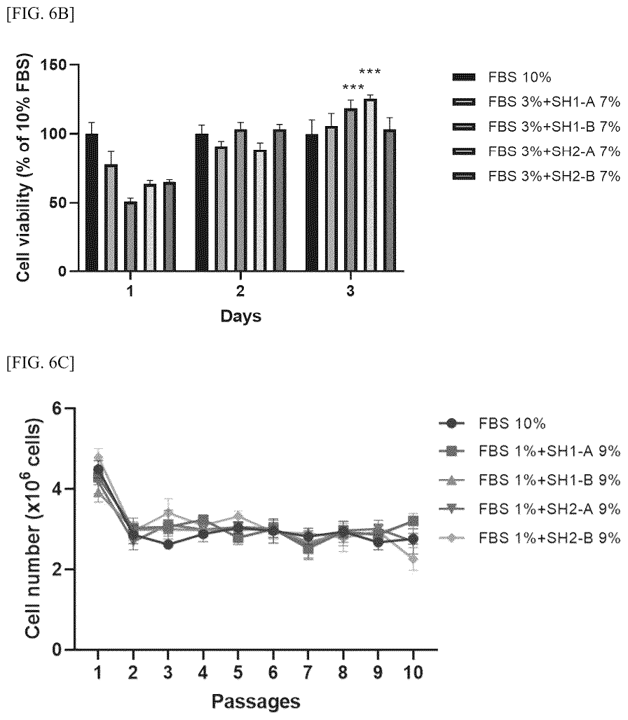

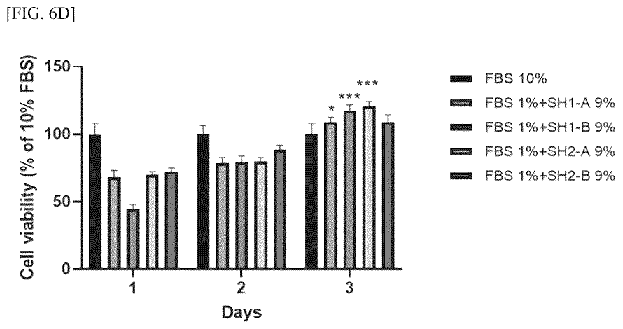

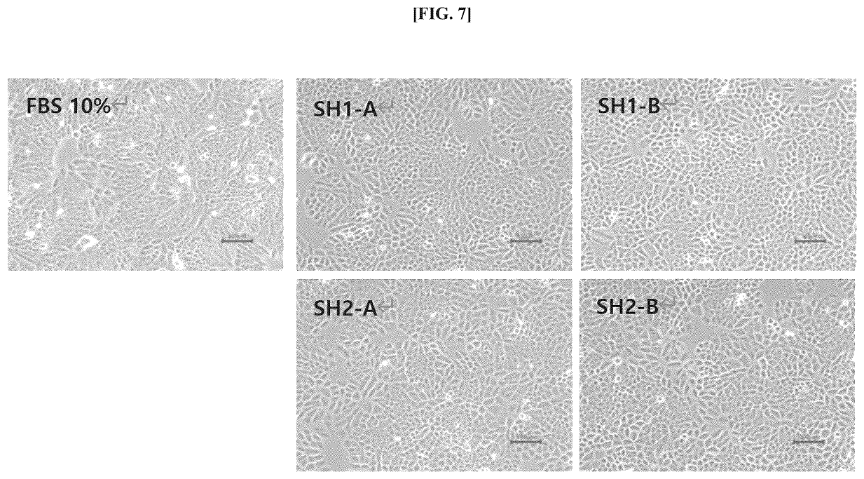

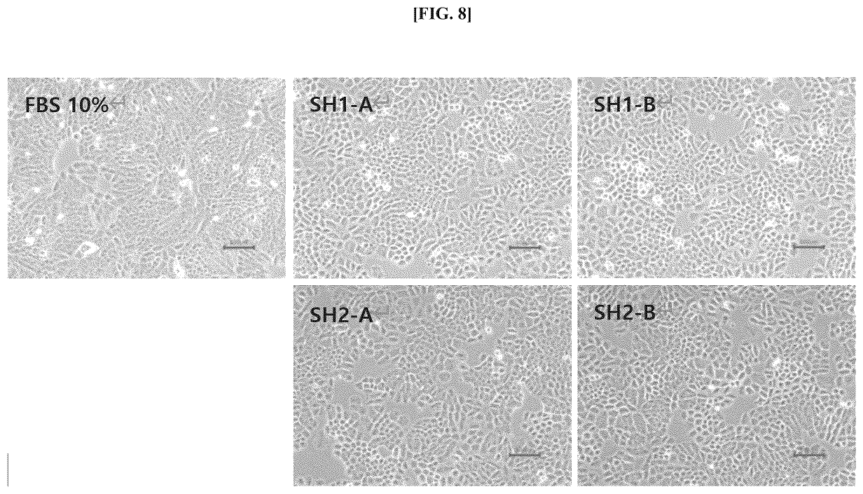

Referring to FIGS. 6A to 6B, 3% FBS and 7% SH1 and SH2 hydrolysates were added to the culture medium to culture H460. As a result, all experimental groups showed higher cell growth rates than that of the control group added with 10% FBS (FIG. 6A) and also showed higher cell survival rate than that of the control group from the 3rd day. When added with 20 g/L of 7% SH1 stock (SH1-B) and added with 10 g/L of 7% SH2 stock (SH2-A), the cell survival rate was highest (FIG. 6D). The morphology of the cultured cells was similar to that of the control group (FIG. 7).

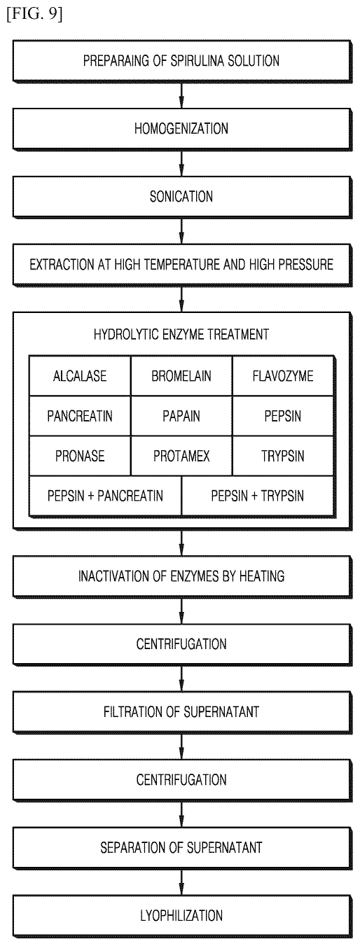

Referring to FIGS. 6C and 6D, 1% FBS and 9% SH1 and SH2 hydrolysates were added to the culture medium to culture H460. As a result, the medium added with 10 g/L of SH2 stock (SH2-A) showed a cell growth rate similar to that of the control group added with 10% FBS, and the medium added with 20 g/L of SH2 stock (SH-2B showed a cell growth rate slightly lower than that of the control group added with 10% FBS. On the other hand, in the medium added with 9% SH1 hydrolysate stocks at the concentrations of 10 g/L of SH1 (SH1-A) and 20 g/L of SH1 (SH1-B), all experimental groups showed higher cell growth rates than that of the medium added with 10% FBS (FIG. 6C). All experimental groups showed higher cell growth rates than that of the control group added with 10% FBS, and showed the highest cell survival rate when 20 g/L of 9% stock (SH1-B) and 10 g/L of 9% stock (SH2-A) were added (FIG. 6D). No. morphological changes in cultured cells was observed either (FIG. 8).

In the medium added with 3% FBS, both media added with 10 g/L and 20 g/L of stock showed a higher cell growth rate than that of the medium added with 10% FBS. All experimental groups did not show any significant difference in the cell growth rate even in the medium added with 1% FBS. Thus, the stock was prepared at the lowest concentration, 10 g/L, and used in subsequent experiments. Since there was no significant difference in the cell growth rate according to the SH1 and SH2 extraction methods as well, the SH1 extract was used to conduct the subsequent experiments.

### Embodiment 6

### Comparison of Cell Growth Rates According to Hydrolytic Enzyme

Cell growth proliferation effects of hydrolysates according to a hydrolytic enzyme were compared. As shown in FIG. 9, in order to compare the cell proliferation ability of the hydrolysates according to a hydrolytic enzyme, as in Embodiment 1, 4% Spirulina solution was homogenized and sonicated for 30 minutes to disrupt the cells, and heat and pressure were applied at 121° C. for 30 minutes to prepare an extract (SH-1), which was then treated with alcalase (50° C., pH 8) (Sigma Aldrich), bromelain (50° C., pH 6) (Sigma Aldrich), flavourzyme (50° C., pH 7) (Sigma Aldrich), pancreatin (37° C., pH 7) (Sigma Aldrich), papain (60° C., pH 7) (Sigma Aldrich), pepsin (37° C., pH 2) (Sigma Aldrich), pronase (37° C., pH 8) (Sigma Aldrich), protamex (50° C., pH 7) (Sigma Aldrich), trypsin)(37° C., pH 8) (Sigma Aldrich) each for 2 hours to prepare 9 hydrolysates. The extract was sequentially treated with pepsin and pancreatin for 2 hours to prepare a hydrolysate, and the extract was sequentially treated with pepsin and trypsin for 2 hours to prepare a hydrolysate. Therefore, a total of 11 hydrolysates were prepared.

After the enzymatic reaction, as in Embodiment 1, the hydrolysates were centrifuged at 9000 rpm for 20 minutes, the supernatant was filtered with 1 μm filter paper, and then centrifuged at 30000 rpm for 20 minutes to separate the supernatant and lyophilize same. The hydrolysates were dissolved in distilled water before use in the experiment, filtered through a 0.2 pm PES filter to remove various contaminants, and then added to the medium for use.

In Embodiment 5, it was confirmed that H460 cells in the medium added with 1% FBS and 9% Spirulina hydrolysate exhibited a cell growth rate similar to that of the medium added with 10% FBS. In order to confirm the cell growth promoting effect of the hydrolysates according to a hydrolytic enzyme, based on the above-mentioned results, 9% of the 11 hydrolysates prepared by the combination of hydrolytic enzymes were added to the medium added with 1% FBS, and the H460 cell line was cultured for 6 generations, and, thereafter, the cell growth rate was measured.

Referring to FIG. 10, H460 cells in the hydrolysate treated with Pepsin and Pancreatin together showed the highest cell growth rate of 106%, as compared to that of the control group added with 10% FBS, and the cell growth rate was higher in the order of pepsin, pepsin+trypsin, pronase, and papain hydrolysate.

### Embodiment 7

### Preparation of Three Hydrolysates Through Combination of Hydrolytic Enzymes

In order to optimize the conditions for hydrolysis of additives promoting cell proliferation, three hydrolysates were prepared by using a combination of pepsin, trypsin, and papain hydrolytic enzymes, which showed high cell proliferation effects through the above-mentioned embodiments.

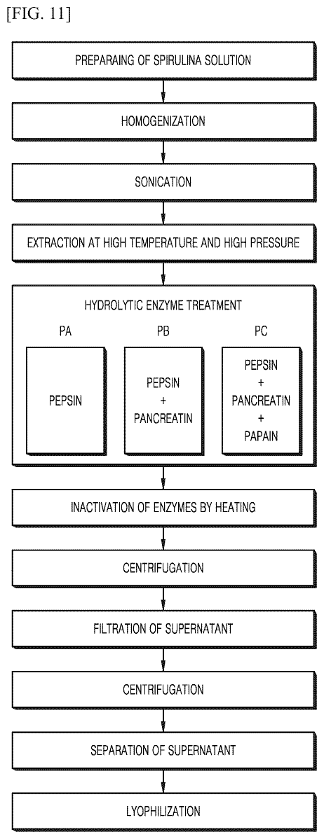

Specifically, as shown in FIG. 11, Spirulina extract SH1 was prepared and sequentially treated with pepsin (37° C., pH 2), pancreatin (37° C., pH 7) and papain (60° C., pH 7) for 2 hours to prepare a total of three hydrolysates, hydrolysate (PA) treated only with pepsin, hydrolysate (PB) treated with pepsin and pancreatin, and hydrolysate (PC) treated with pepsin, pancreatin and papain. After each enzyme reaction, the enzyme was inactivated by boiling at 95° C. for 10 minutes, centrifuged at 9,000 rpm for 20 minutes, and the supernatant was filtered with 1 μm filter paper and centrifuged at 30,000 rpm for 20 minutes to obtain the supernatant and then lyophilized. The lyophilized hydrolysate powder was dissolved in distilled water at a concentration of 10 g/L before use in cell culture, and then filtered through a 0.2 μm PES filter to remove various contaminants to prepare a stock, which was added to the medium and used.

### Embodiment 8

### Preparation of Three Hydrolysates Through Combination of Hydrolytic Enzymes

After the SH1 extraction process, hydrolysates (PA, PB, PC) prepared through the combination of three hydrolytic enzyme were added to the media to culture the H460 cell line, and the cell growth rate and cell survival rate were measured. The media added with 5%, 3% and 1% FBS were added with 5%, 7% and 9% hydrolysates, respectively, and used for cell culture.

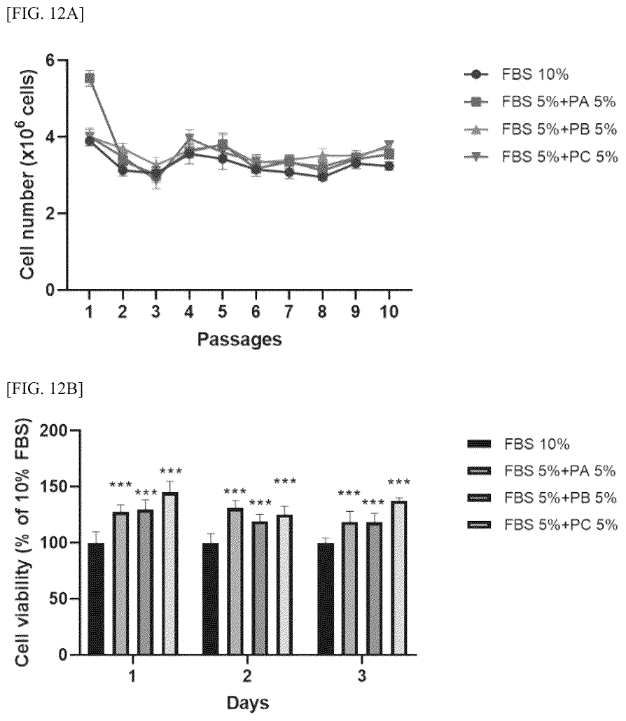

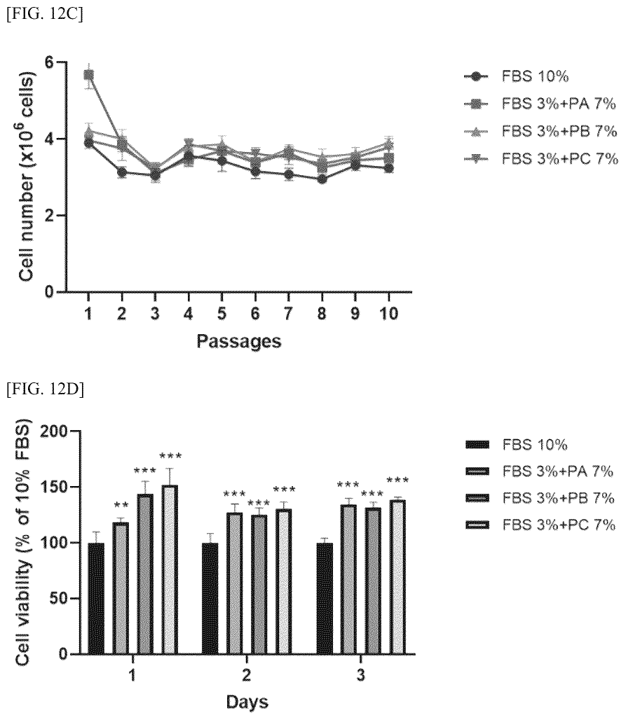

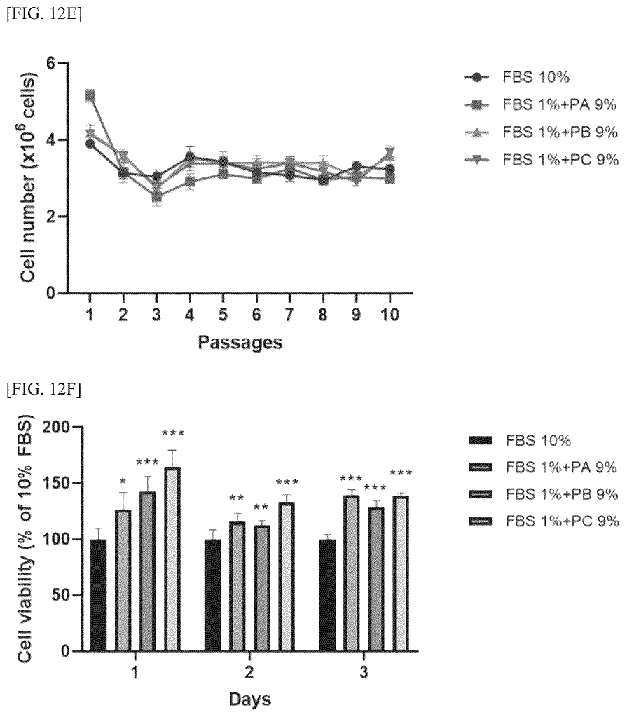

FIGS. 12A to 12F show the cell growth rate and cell survival rate of the H460 cell line. When 5% FBS medium was cultured by adding 5% hydrolysate thereto, the PB and PC groups showed the cell growth rates of 110% and 114%, higher than that of 10% FBS medium (FIG. 12A), and the PA and PC groups showed the highest cell survival rate of 139%, as compared to that of 10% FBS medium (FIG. 12B).

In addition, when 3% FBS medium was cultured by adding 7% of hydrolysate thereto 3% FBS medium, the PB and PC groups showed the growth rates of 121% and 117%, higher than that of 10% FBS medium (FIG. 12C), and PA, PB, and PC all exhibited very high cell survival rate rates of 130% or more, as compared to that of the control group (FIG. 12D).

In addition, even when the FBS 1% medium was cultured by adding 9% hydrolysate thereto, the PB and PC groups showed the cell growth rates of 110% and 114%, higher than that of the control group added with 10% FBS (FIG. 12E). The group added with all hydrolysates showed a higher cell survival rate than that of the control group. In particular, the PA and PC groups showed the highest rate of 139%, as compared to that of the control group (FIG. 12F).

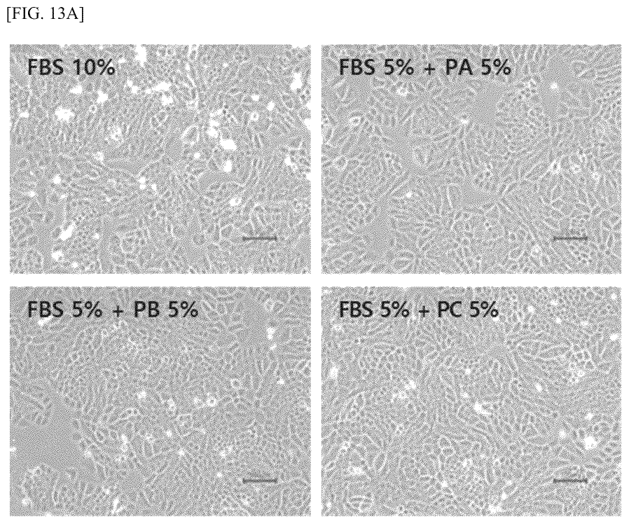

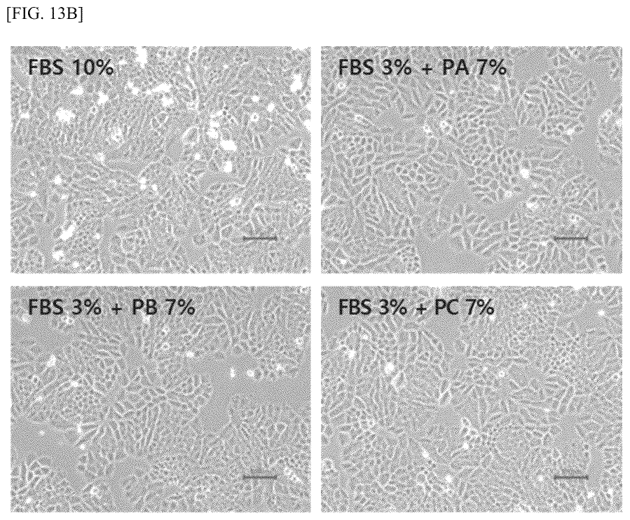

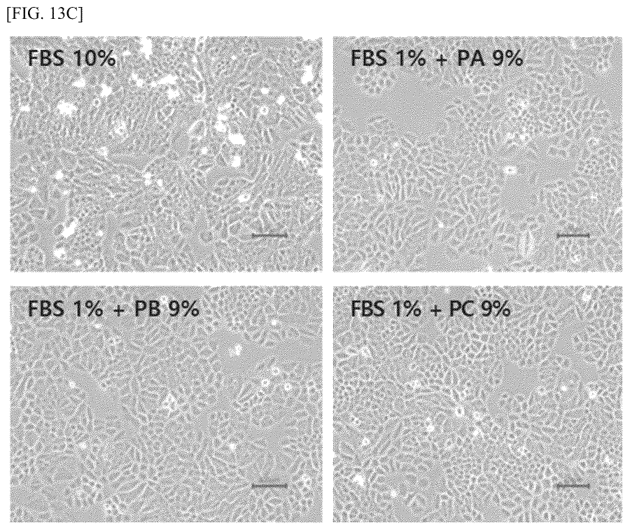

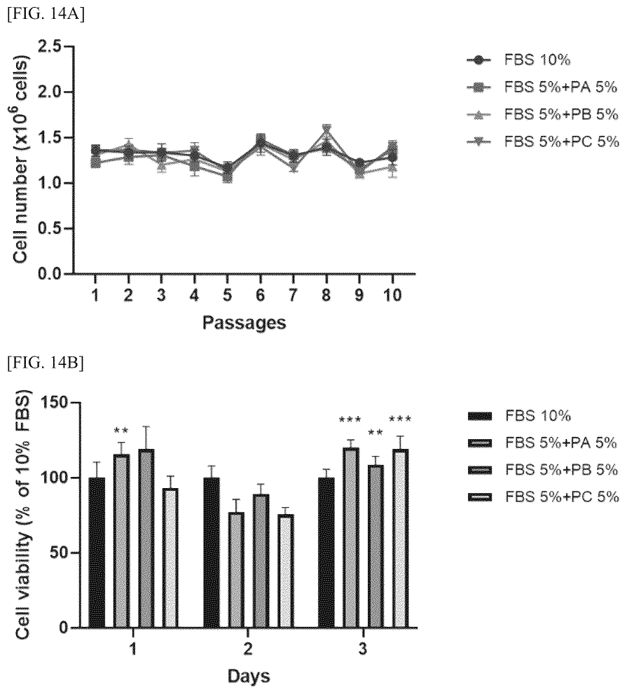

Referring to FIGS. 13A to 13C, no morphological changes of the H460 cell line was observed in all groups cultured by adding 5%, 3%, and 1% of FBS and adding 5%, 7%, and 9% of hydrolysates PA, PB, and PC, and the morphology was similar to that of the control group.

### Embodiment 9

### Hela Cell Proliferation Efficacy of Spirulina Hydrolysates

As in Embodiment 8, Spirulina hydrolysates (PA, PB, PC) were added to the media to culture the Hela cell line, and the cell growth rate and cell survival rate were measured. HeLa was cultured at 37° C. and with 5% CO₂ using Minimum Essential Medium (MEM) (Sigma Aldrich) medium.

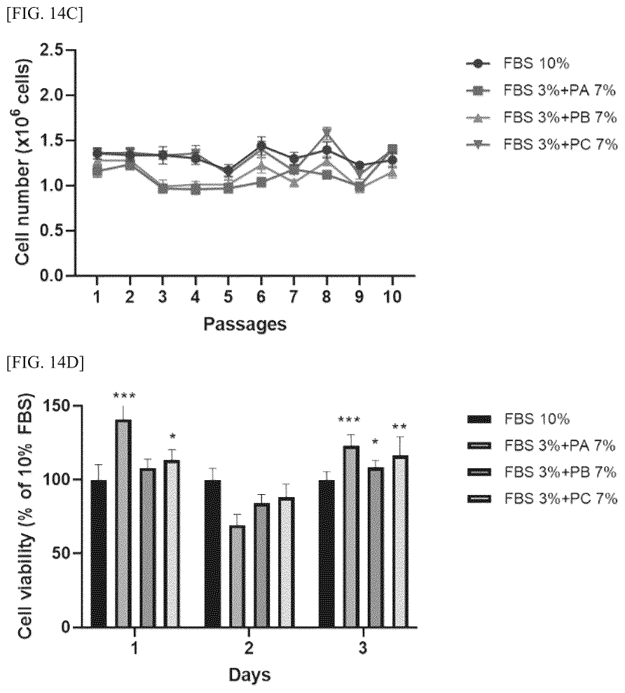

FIGS. 14A to 14D show the cell growth rate and cell survival rate of Hela cell line. When 5% FBS medium was cultured by adding 5% hydrolysate thereto, the PA and PC groups showed the cell growth rates of 106% and 109%, respectively, higher than that of 10% FBS medium (FIG. 14A). The PA and PC groups also showed the cell viabilities of 120% and 119%, respectively, higher than that of the control group (FIG. 14B).

In addition, when 3% FBS medium was cultured by adding 7% hydrolysate thereto, the PA and PC hydrolysate groups showed the cell growth rate of 109%, higher than that of 10% FBS medium (FIG. 14C). The groups cultured in media added with PA and PC exhibited the cell survival rate rates of 123% and 116%, respectively, higher than that of 10% FBS medium (FIG. 14D).

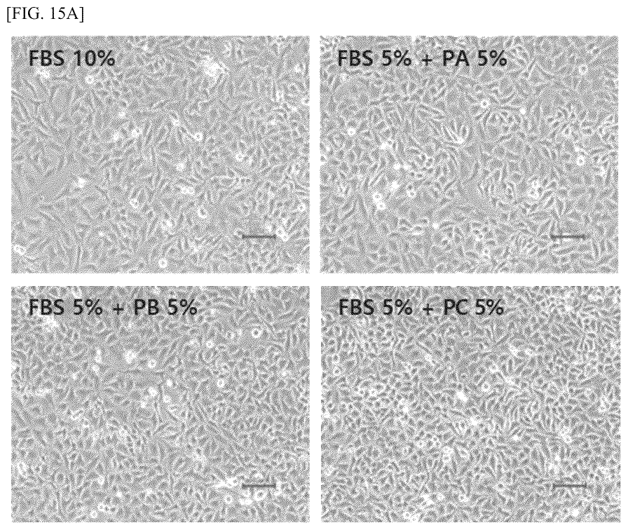

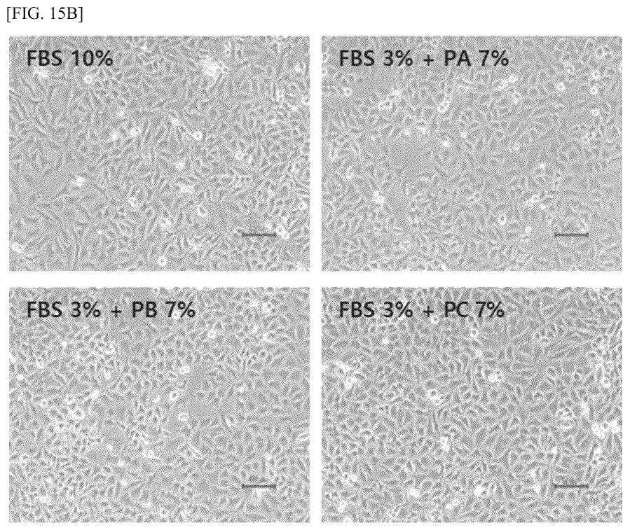

Referring to FIGS. 15A and 15B, no specific morphological changes of the Hela cell line was observed in all groups cultured by adding 5% and 3% of FBS and 5% and 7% of hydrolysates PA, PB and PC, and the morphology was similar to that of the control group.

Embodiments about the cell culture medium composition and method for producing same according to the present disclosure were described above, but it is apparent that various modifications may be achieved without departing from the scope of the present disclosure.

Therefore, the scope of the present disclosure should not be limited to the embodiment(s) and should be determined by not only the following claims, but equivalents of the claims.

That is, it should be understood that the embodiments described above are not limitative, but only examples in all respects, the scope of the present disclosure is expressed by claims described below, not the detailed description, and it should be construed that all of changes and modifications achieved from the meanings and scope of claims and equivalent concept are included in the scope of the present disclosure.

### INDUSTRIAL APPLICABILITY

The present disclosure relates to a cell culture medium composition and a preparation method therefor. By replacing an animal serum with a Spirulina hydrolysate, cell growth can be induced and cell proliferation can be promoted to a level greater than or equal to that of a medium containing an animal serum, while significantly reducing the added amount of animal serum.

1. A method for producing a cell culture medium composition, the method comprising:
a first step of obtaining a Spirulina extract from Spirulina;
a second step of treating the Spirulina extract with a hydrolytic enzyme to prepare a Spirulina hydrolysate; and
a third step of filtering and recovering the Spirulina hydrolysate.

2. The method for producing a cell culture medium composition of claim 1, wherein the Spirulina is at least one selected from the group consisting of Spirulina maxima, Spirulina platensis, Spirulina geitleri, Spirulina siamese, Spirulina major, Spirulina subsalsa, Spirulina princeps, Spirulina laxissima, Spirulina curta, and Spirulina spirulinoides.

3. The method for producing a cell culture medium composition of claim 1, wherein the first step is, after homogenizing the Spirulina solution, (a) sonicating, followed by extracting at a high temperature and a high pressure, or (b) freeze-thaw dissolving, followed by extracting by sonication.

4. The method for producing a cell culture medium composition of claim 1, wherein the hydrolytic enzyme of the second step has proteolysis.

5. The method for producing a cell culture medium composition of claim 1, wherein the hydrolytic enzyme of the second step is used alone or in combination of two or more.

6. The method for producing a cell culture medium composition of claim 5, wherein two or more kinds of the hydrolytic enzyme is used sequentially.

7. The method for producing a cell culture medium composition of claim 1, wherein the hydrolytic enzyme of the second step is selected from the group consisting of Alcalase, bromelain, flavourzyme, pancreatin, papain, pepsin, pronase, protamex, trypsin, or a combination thereof.

8. The method for producing a cell culture medium composition of claim 1, wherein the third step is centrifuging the Spirulina hydrolysate, filtering the supernatant, centrifuging the filtrate, separating the supernatant and lyophilizing the same.

9. A cell culture medium composition comprising a Spirulina hydrolysate as an active ingredient.

10. The cell culture medium composition of claim 9, wherein the hydrolysate is obtained by treating the Spirulina extract with a proteolytic enzyme.

11. The cell culture medium composition of claim 9, wherein the proteolytic enzyme is selected from the group consisting of pepsin, pancreatin, trypsin, pronase, papain, or a combination thereof.

12. The cell culture medium composition of claim 9, wherein the Spirulina hydrolysate has a concentration of 5 g/L or less.

13. The cell culture medium composition of claim 12, wherein the composition comprises the Spirulina hydrolysate in an amount of 0.1 to 20 vol % (v/v).

14. The cell culture medium composition of claim 12, wherein the composition comprises serum and Spirulina hydrolysate in an amount of 0.1 to 20 vol % (v/v).

15. The cell culture medium composition of claim 14, wherein the composition comprises serum in an amount of 0.1 to 19.9 vol % (v/v).

16. The cell culture medium composition of claim 14, wherein the composition comprises serum and Spirulina hydrolysate in a volume ratio (v/v) of 5:5 to 1:9.

17. The cell culture medium composition of claim 9, wherein the cells are animal cells, insect cells or plant cells.
